(19) United States
(12) Patent Application Publication
(10) Pub. No.: US 2004/0014726 A1
(43) Pub. Date: Jan. 22, 2004

(54) Method for the preparation of a pharmaceutical composition, and its use

(76) Inventors: Thua Osterman, Turku (FI); Ritva Hannuniemi, Kuusisto (FI); Teuvo Hentunen, Turku (FI); Sirpa Liukko-Sipi, Vinkkila (FI); Hannu Nikander, Paattinen (FI); Katri Selander, Birmingham, AL (US); Raija Sellman, Turku (FI); Kalervo Vaananen, Turku (FI)

Correspondence Address: BIRCH STEWART KOLASCH & BIRCH, PO BOX 747, FALLS CHURCH, VA, 22040-0747, US

(21) Appl. No.: 10/415,085

(22) PCT Filed: Oct. 26, 2001
(86) PCT No.: PCT/FI01/00929

(30) Foreign Application Priority Data
Oct. 27, 2000 (FI) 20002361
Oct. 27, 2000 (FI) 20002362

(51) Int. Cl. A61K 31/66

(52) U.S. Cl. 514/102

(57) ABSTRACT

The present invention is directed to a method for the preparation of a pharmaceutical composition having osteoclast formation inhibiting or suppressing activity, characterized in that, as the active agent, a compound of the formula (I) is used, in which formula Q¹ is hydrogen, hydroxyl, halogen, amino —NH2, or —OR′, wherein R′ is C1-C4-alkyl or acyl, Q² is straight or branched alkyl, hydroxyalkyl or aminoalkyl, or a corresponding unsaturated group optionally substituted at the hydroxy or amino group, or a unsubstituted or substituted, saturated or unsaturated cycloalkyl, which can be bound to the methylene carbon over a straight or branched alkylene group, or is the group (a) wherein Y is a saturated or unsaturated heterocyclic group, or a carbocyclic aromatic group, X is a bond, O, S or NR³, wherein R³ is hydrogen or a protecting group, especially lower alkyl, acyl, aryl or arylalkyl, and n is an integer 0 to 6, R¹ and R² are independently hydrogen or C1-C4-alkyl, or it is halogen, and the groups R1 to R4 are the same or different, and mean an alkyl group with 1-5 carbon atoms, whereby one or two of the groups R1 to R4 can also mean hydrogen, or a pharmacologically acceptable salt thereof.

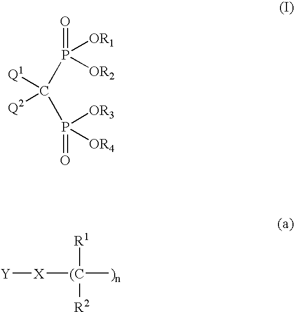

### FIELD OF THE INVENTION

The present invention relates to the preparation of pharmaceutical compositions with osteoclast formation (osteoclastogenesis) inhibiting or suppressing activity. The osteoclast formation inhibiting activity manifests itself especially as an effect in strengthening cortical bone. The present invention also relates to the use of such a pharmaceutical composition, as such or in combination with other bone active treatments including, but not limited to, bisphosphonates and specific estrogen receptor modifying agents (SERMs).

### BACKGROUND OF THE INVENTION

Bisphosphonates are used as therapeutic agents for the treatment of pathological bone destruction of various origin, such as osteolytic bone disease due to malignancy, Paget's disease, and osteoporosis. They are analogues of the physiologically occurring inorganic pyrophosphates. The basic P-C-P structure of the bisphosphonates makes it possible to design a great number of different compounds by changing the side chains of the carbon atom. A different approach is to form various derivatives of the phosphate groups.

In general, bisphosphonates inhibit osteoclast activity, that is of the cells which are responsible for bone resorption. Known bisphosphonates bound to the bone matrix, enter resorbing osteoclasts and reduce the activity of matured osteoclasts. They inhibit bone resorption both in vitro and in vivo. Limited absorption from the gastrointestinal tract, fast incorporation in bone tissue, and unchanged excretion in urine are all characteristics of the known bisphosphonates.

In the patents U.S. Pat. No. 4,447,256, DE 28 31 578, JP 55089210, JP 55098105, JP 55043054, JP 55043055 processes are described for the preparation of some pyridinylaminomethylene bisphosphonic acid tetraalkylesters. The disclosed compounds are suggested to be used as herbicides.

U.S. Pat. No. 5,866,556 on the other hand discloses pyridinylaminomethylene bisphosphonic acid tetraalkylesters with a high oral bioavailability and practically no affinity to bone. According to this patent, the disclosed compounds are said to have less side effects without a loss of their antiresorptive activity. Also EP patents 563107 and 563096 disclose substituted methylene bisphosphonic acid in partial ester form for the treatment of various bone diseases, such as for the prevention of bone resorption and osteoporosis.

According to the invention it has now been discovered that a group of methylene bisphosphonic acid esters exhibits an inhibitory or suppressing effect on osteoclast formation, that is they affect osteoclast formation at an early stage of this osteoclastogenesis process but do not have a direct effect on the bone.

According to the invention it has been found that it is possible to suppress the osteoclastic activity at an early stage of the osteoclast formation process, rather than to exert an effect on the mature osteoclast. This is clearly evidenced as a strengthening effect of specifically the cortical bone. An effect is also seen in the density of the trabecular bone as a result of treatment according to the invention, but as far as the overall strengthening of the bone is concerned, a strengthening of the denser cortical bone is more important than a strengthening of the trabecular bone. The invention is especially useful for treatment to combat weakening of cortical bone, in addition to trabecular bone, in a postmenopausal female, a child, an elderly male, in a patient treated with corticosteroids, cancer chemotherapies or other bone strength weakening therapies, in a patient with cancer metastases in the bone, and in a cancer patient whose bone strength is weakened by local or systemic factors (cytokines etc.) secreted by the cancer cells.

The finding that has been made in the course of the invention can be applied in the treatment or prevention of a variety of bone diseases or conditions, including, but not limited to, the use for strengthening of cortical bone. Other conceivable indications where it is possible to take advantage of the long-term inhibitory activity on osteoclast formation include juvenile osteoporosis, juvenile rheumatism, osteogenesis imperfecta, local bone disease such as vanishing bone disease etc. The invention is also useful for preventing formation and growth of bone metastates, for which weakening of cortical and trabecular bone is known to be a prerequisite.

It has also been established that the active agent used does not bind to bone, as a consequence of which it is not retained in the organism, which fact is important from the point of view of side-effects. Further, the compounds used do not inhibit remodelation of the bone, i.e. the normal regeneration of the bone wherein part of the bone is resorbed and new bone is formed. This is especially important in the treatment of children and fertile women.

Known bisphosphonates bind to the bone and are retained there even for long periods of time. They also inhibit all bone resorptions, including the normal remodelation of the bone. Consequently, they cannot be used for example in the treatment of children and fertile women.

The present invention is thus directed to a method for the preparation of a pharmaceutical composition with osteoclast formation inhibiting or suppressing activity, which method is characterized in that a compound having the general formula

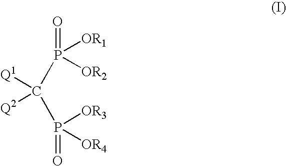

is used, in which formula

Q¹ is hydrogen, hydroxyl, halogen, amino —NH₂, or —OR′, wherein R′ is C₁-C₄-alkyl or acyl,

Q² is

a) straight or branched C₁-C₁₀-alkyl, -hydroxyalkyl or -aminoalkyl, or C₂-C₁₀-alkenyl, -hydroxyalkenyl or -aminoalkenyl, or -alkynyl, -hydroxyalkynyl or -aminoalkynyl, whereby the oxygen may, as a substituent, carry one protecting group, and the nitrogen, as a substituent, may carry one or two protecting groups, especially C₁-C₆-alkyl, acyl, aryl or arylalkyl, or Q² is unsubstituted or substituted, saturated or unsaturated C₃-C₁₀-cycloalkyl, which can be bound to the methylene carbon over a straight or branched C₁-C₄-alkylene group, or

b) the group

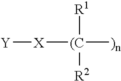

wherein Y is a saturated, partly saturated or an aromatic heterocyclic group, or a carbocyclic aromatic group, whereby the heterocyclic group can contain 1 to 3 hetero atoms selected from the group N, O and S, X is a bond, O, S or —NR³, wherein R³ is hydrogen or a protecting group, especially C₁-C₆-alkyl, acyl, aryl or arylalkyl, and n is an integer 0 to 6, R¹ and R² are independently hydrogen or C₁-C₄-alkyl, or

c) halogen,

and the groups R₁ to R₄ are the same or different, and mean an alkyl group with 1-5 carbon atoms, whereby one or two of the groups R₁ to R₄ can also mean hydrogen, or a pharmacologically acceptable salt thereof.

In this description, cycloalkyl is preferably a saturated C₃-C₇-cycloalkyl group, such as cyclopropyl, cyclobutyl, cyclopentyl, cyclohexyl or cycloheptyl. The cycloalkyl group can be substituted with 1 to 3 substituents, preferred substituents being hydroxy, C₁-C₄-alkyl, -alkoxy, halogen, amino and/or nitro.

The saturated or partly unsaturated or aromatic heterocyclic ring in the meaning of Y can be unsubstituted or substituted, the substituents being the same as mentioned for cycloalkyl above. Such a group is e.g. morpholinyl, thiomorpholinyl, oxathiane, dithiane, piperidinyl, piperazinyl, azetidinyl, pyrrolidinyl, pyrrolyl, imidazolyl, triazolyl, oxazolyl, thiazolyl, pyridinyl, pyrimidinyl, pyrazinyl, pyridazinyl or azepinyl. Preferably it contains 3 to 6 ring atoms, and especially it is a pyridinyl, pyrrolidinyl, piperidinyl, pyrimidinyl, or phtalimidyl. These heterocyclic groups may be substituted as has been described for cycloalkyl above.

A carbocyclic aromatic group Y is a substituted or unsubstituted aromatic cyclic group, such as a monocyclic aryl or aralkyl, especially phenyl, or a conjugated or bridged unsaturated or partly saturated ring system, such as naphtyl, phenanthryl, indenyl, indanyl, tetrahydronaphtyl, biphenyl, di- and triphenyl methyl, etc. These groups may be substituted as has been described for cycloalkyl above.

A monocyclic aryl or aralkyl is preferably phenyl or phenyl-C₁-C₆-alkyl, which is optionally substituted with 1 to 3 groups selected from hydroxy, C₁-C₄-alkyl, -alkoxy, halogen, amino and/or nitro.

Acyl can be alkyl-, aryl- or arylalkylcarbonyl, but also alkoxy-, aryloxy- or aralkoxycarbonyl, wherein alkyl contains 1 to 4 carbon atoms, and aryl has the meaning given above, especially phenyl.

Halogen means chlorine, bromine, fluorine or iodine.

In the above definitions as well as in the definitions below, a hydroxy and an amino group can be either in free form or in the form of a protected group. The term “protecting group” is well known in the art and refers to a large number of groups suitable for protecting a hydroxyl and/or an amine function in a molecule from undergoing chemical reactions, yet being readily removed after a desired chemical reaction has taken place. A “protecting group” can also be used to derivatize the original molecule via an amino or hydroxyl group, e.g. in order to modify physical, chemical or pharmacological properties of the molecule. The choice of this “protecting group” depends on what kind of properties are desired, whether a hydroxy or an amino group is being protected, subsequent reaction conditions, and conditions for removal, which choice is within the ability of one skilled in the art. If a protecting group is to be removed, the most useful protecting groups in this invention are those which may be removed either with the desired or without any effect on the phosphorus moyeties.

Examples of suitable protecting groups are described e.g. by T. W. Greene, “Protective Groups in Organic Synthesis”, John Wiley & Sons, Inc., 1981, Houben-Weyl, Methoden der organischen Chemie, Georg Thieme Verlag; Protective Groups in Organic Synthesis, John Wiley & Sons, New York, 1980; Greene and Wuts “Protective Groups in Organic Synthesis”, 2d. Ed. John Wiley & Sons, Inc., New York, 1991.

Without limiting the scope of the present invention, preferable protecting groups for hydroxyl include for example unsubstituted or substituted alkyl groups with 1 to 8 carbon atoms, such as methyl or ethyl, cycloalkyls, silyls, aryl or alkylsilyls, such as trimethylsilyl or t-butyldimethylsilyl, substituted or unsubstituted aryls or arylalkyls, such as the benzyl group, diphenylnethyl or triphenylmethyl, substituted or unsubstituted tetrahydropyranyls, substituted or unsubstituted alkanoyls or alkanoylalkyls, such as e.g. acetyl or pivaloylmethyl, substituted or unsubstituted aroyls or aroylalkyls, such as benzoyl, alkylsulfonyls, arylsulfonyls, residues of heterocyclic acids, alkoxycarbonyls, alkoxyalkoxycarbonyls, aryloxycarbonyls, alkyloxyalkyls, aryloxyalkyls, ketals or acetals, including cyclic ketals and acetals.

Useful amino protecting groups include for example alkyl groups with 1 to 8 carbon atoms, substituted or unsubstituted aryls or arylalkyls, such as a benzyl group, alkanoyls, such as acetyl, alkanoylalkyls, substituted or unsubstituted aroyl or aroylalkyl groups, phthalyls, cycloalkyls, silyls, aryl or alkylsilyls, such as trimethylsilyl or t-butyldimethylsilyl, alkyloxyalkyls, aryloxyalkyls, alkoxycarbonyls, e.g. methoxycarbonyl, tert.-butoxycarbonyl, aryloxycarbonyls or substituted aryloxycarbonyls like benzyloxycarbonyl or fluorenylmethoxycarbonyl. In all the above definitions for protecting groups, alkyl as such or as a part of another group, such as in alkanoyl or alkoxy, means a group containing from 1 to 8 carbon atoms. Cycloalkyl contains preferably 3 to 10 carbon atoms. Any substituents are the same as those referred to above.

According to a preferred embodiment, where Q² has the meaning defined under b), there is always present at least one heteroatom selected from the group of O, N and S, either in the ring Y and/or in the bridging group X.

According to an advantageous embodiment, Q¹ is hydrogen or hydroxyl.

According to a another preferred embodiment, Q² is C₁-C₆-alkyl or hydroxy- or amino-substituted C₁-C₆-alkyl, such as methyl, ethyl, propyl, isopropyl, straight or branched butyl or pentyl, or the corresponding hydroxy- or amino-substituted group, such as 2-hydroxy- or 2-amino-ethyl, 3-hydroxy- or 3-amino-propyl, 4-hydroxy- or 4-amino-butyl, 5-hydroxy- or 5-amino-pentyl, or C₁-C₆-alkyl-mono- or -di-substituted amino-C₁-C₆-alkyl, especially -C₁-C₄-alkyl.

According to a further embodiment, Y is a nitrogen-containing heterocyclic group, such as pyridinyl, pyrimidinyl or piperidinyl, especially pyridinyl, which can be substituted with 1 to 3 substituents selected from hydroxy, protected hydroxy, C₁-C₄-alkyl, C₁-C₄-alkoxy, halogen or nitro. In these compounds, the group X is preferably —NH—, and n is 0.

Among the compounds referred to above, the triesters and especially the tetraesters are the most preferred.

An especially preferred group is formed by the group of compounds having the formula

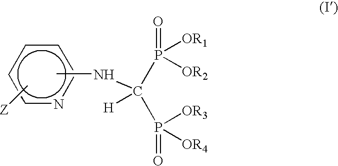

wherein Z has the meaning of hydroxy or protected hydroxy, and the groups R₁ to R₄ are the same or different, and mean an alkyl group with 1-5 carbon atoms, whereby one of the groups R₁ to R₄ can also mean hydrogen.

A protected hydroxy group as Z is preferably a lower alkoxy group with 1 to 4 carbon atoms, an aryloxy group, such as a benzyloxy group, or an acyloxy group, such as a lower alkyl acyloxy group, for example an acetoxy group, a pivaloyloxy-, aryl- or alkylsilyloxy group.

Z in the above mentioned formula is preferably hydroxy, and the groups R₁ to R₄ are the same, and are preferably ethyl groups. Such a compound is especially [[(3-hydroxy-2-pyridinyl)amino]methylidene]bisphosphonic acid tetraethylester.

The said compounds as well as their manufacture is as such known, see for example U.S. Pat. No. 5,866,556, EP 563107 and 563096.

The pharmaceutical compositions made according to the invention may be used to treat any condition which is responsive to the effect of an osteoclast formation inhibiting or suppressing agent. Such compositions can be administered for example orally, parenterally, topically or rectally by means of any pharmaceutical formulation useful for said administration, and containing the active agent in a therapeutically effective and acceptable amount together with pharmaceutically acceptable carriers, adjuvants or vehicles known in the art. The manufacture of such pharmaceutical formulations is well known in the art.

Thus the pharmaceutical composition may be in a dosage form suitable for oral use, such as tablets, capsules, liquid dosage forms, such as solutions, suspensions, emulsions, syrups, gels etc. All such formulations are made using per se known formulation on techniques as well as known carriers, adjuvants and additives. Suitable excipients for oral administration forms are fillers, those for example for use in tablets including sugars, such as lactose, mannitol, sucrose, cellulose preparations, such as starch, gelatins, methyl cellulose, hydroxypropyl cellulose, microcrystalline cellulose, or silicified microcrystalline cellulose, etc.

The compounds according to the invention may also be administered parenterally, for example as infusions or injections, using aqueous or oily suspensions, emulsions, or dispersions containing the active agent in combination with conventional pharmaceutically acceptable excipients, such as buffers, suspension and/or dispersion agents, preservatives etc. Also contemplated in the invention are formulations for topical or transdermal use in the form of creams, ointments, jellies, solutions, suspensions or the like, or in the form of transdermal delivery systems, all of which are known to the person skilled in the art. For rectal use, the compounds according to the invention are combined with a substance suitable for rectal administration such as waxes or fats or other known suppository bases. It is also possible to administer the compounds in the form of slow release formulations, the various forms of which are well known in the art. Another mode of administration is in the form of implants, such as subcutaneous implants, or as prosthetic implants, such as by treating the prosthesis with the compound to be administered, or in the form of bone cements.

The therapeutic dose to be given to a patient in need of treatment will vary depending i.a. on the body weight and age of the patient, the particular condition being treated, as well as the manner of administration and are easily determined by a person skilled in the art. A suitable amount can thus vary within wide limits, and can easily be established by a skilled physician. For most purposes an oral dose of appr. 0.01 to 15 mg/kg body weight/day would be suitable, for example when given 1 to 4 times daily. Such a dose means a daily dose of appr. 1 to 1200 mg for a subject weighing 80 kg.

Testing of the Activity

The inhibitory activity of a representative compound to be used according to the invention, namely [[(3-hydroxy-2-pyridinyl)amino]methylidene]bisphosphonic acid tetraethylester (test compound, TC), was tested on different rat and mouse cell cultures, in order to assess its activity as an osteoclast formation inhibitor.

Cultures Used

A. Mouse Bone Marrow Culture

This culture system is the most used method to study osteoclast differentiation (Takahashi et al. 1988a). Mouse marrow cells can be induced to form active osteoclasts in 6-8 days in the presence of an osteotropic factor, such as 1,25(OH)₂D₃, parathyroid hormone, interleukin-1 or prostaglandin E₂ (Takahashi et al. 1988b, Shinar et al. 1990, Collins and Chambers 1992, Jimi et al. 1999). Typically, 10⁶ cells are cultured/ml of media for 6 days. Then, the cells are fixed with 3% paraformaldehyde in PBS and stained for osteoclast marker, such as tartrate-resistant acid phosphatase (TRAP). Sigma's immunohistochemical kit can be used. Multinucleated, TRAP-positive cells containing at least 3 nuclei are counted as osteoclasts (Hentunen et al. 1998).

B. Mouse Spleen Cell Culture

Spleen has been shown to contain a population of early osteoclast precursors that can be induced to differentiate into osteoclasts in the presence of stromal cells, which have the capacity to support osteoclast formation (Udagawa et al. 1989). Without these supporting stromal cells, spleen cells do not differentiate into osteoclasts.

C. Mouse Osteoblast Culture

Bone marrow stromal cells have long been known as a source of osteoprogenitor cells (Owen and Friedenstein 1988). Mouse bone marrow stromal cells are commonly induced to form preosteoblasts by culturing them for 6 days in the presence of glucocorticoid, ascorbic acid and sodium β-glycerophosphate. These preosteoblasts can be further induced to form active osteoblasts by continuing the cultures for two more weeks in the presence of estrogen (17β-estradiol). During this culture period cells begin to express increasing levels of osteoblast markers, such as alkaline phosphatase, type I collagen and osteocalcin, and finally they are able to form bone. Bone formation is detected by bone nodule formation and calcium deposition in the cultures (Qu et al. 1998).

D. Mouse Spleen Cell/Osteoblast Co-Culture

Spleen has been shown to contain a population of early osteoclast precursors that can be induced to differentiate into osteoclasts in the presence of stromal cells, which have the capacity to support osteoclast formation (Udagawa et al. 1989). Without these supporting stromal cells, spleen cells do not differentiate into osteoclasts. A co-culture system was developed, which contains mouse spleen cells and primary mouse osteoprogenitor cells. Osteoprogenitor cells were prepared from mouse marrow as described in section C.

E. Rat Osteoclast Culture

Osteoclasts can be isolated from long bones of several species and they are cultured on bone or dentine slices to study their resorption activity (Boyde et al. 1984, Chambers et al 1984). Mostly, either rat or rabbit osteoclasts are used. They are isolated from new-born animals and cultured for 1-2 days. The resorption activity is determined either by counting the number of resorption pits on bone/dentine slices or the total resorption area is measured using a phase contrast microscope and image analysis system. We are routinely using isolated rat osteoclasts and we culture them on bovine bone slices for two days, after which we fix the cells. Cells are stained with toluidine blue and counted under the microscope (Arnett and Dempster 1987, Lakkakorpi et al. 1989). Then the cells are removed by brushing them and the resorption pits are stained with WGA lectin, which binds to resorption lacuna in the bone slice. The number and area of pits are quantified (Selander et al. 1994).

The concentration range of the test compound in these assays was 10⁻¹⁰-10⁻⁵ M.

Results of the Culture Studies

A. Mouse Bone Marrow Culture

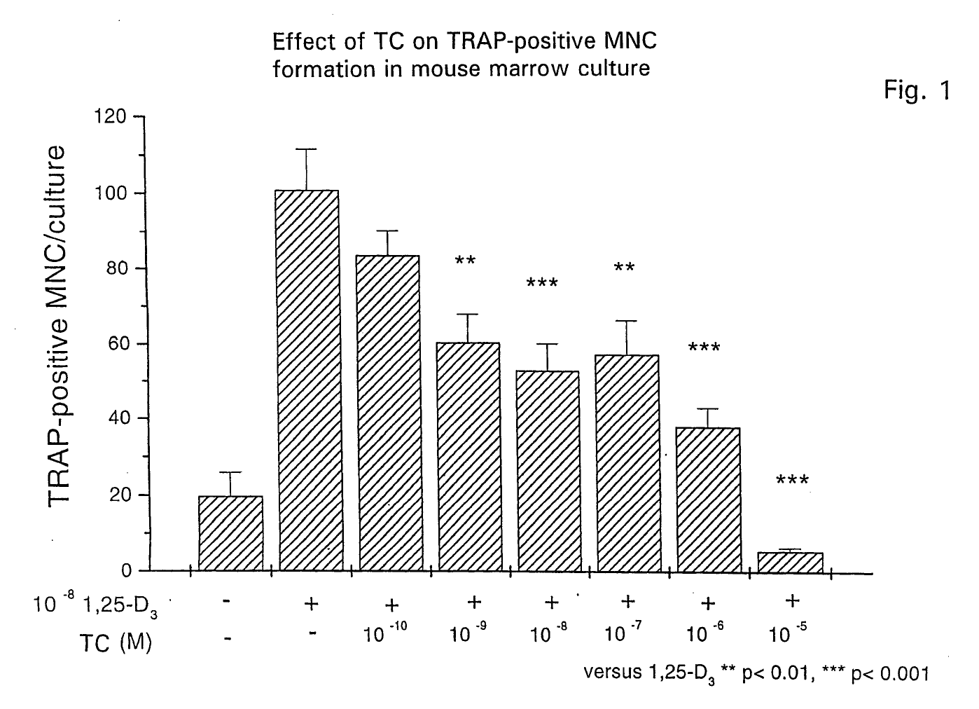

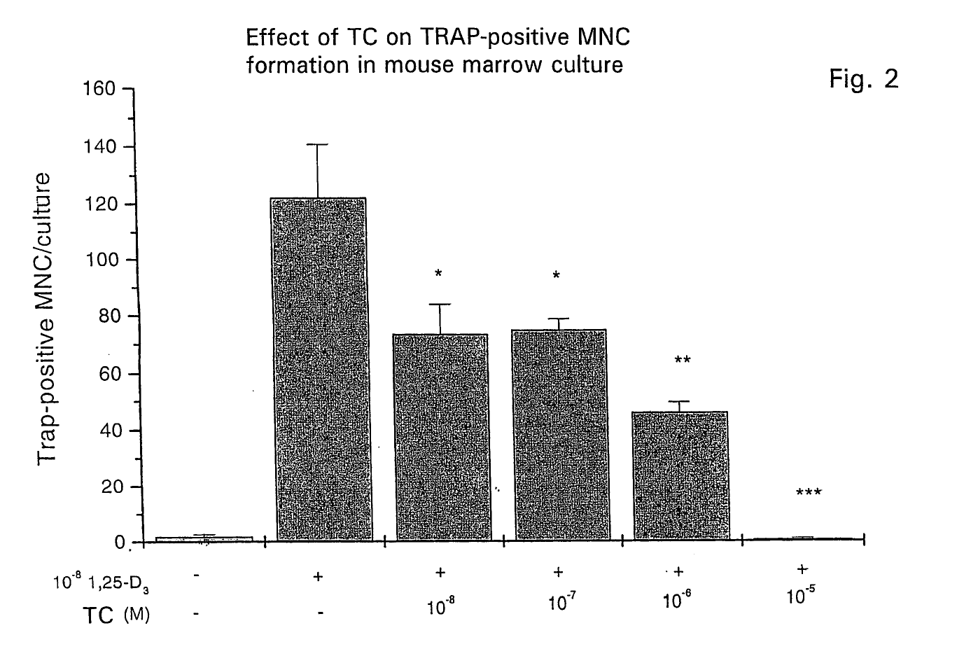

The test compound dose dependently inhibited both 1,25(OH)₂D₃- and PTH-induced osteoclast formation in bone marrow culture. 60% inhibition in osteoclast formation was obtained at 10⁻⁶ M. The test compound at 10⁻⁵ M was found to inhibit completely TRAP-positive MNC formation without having toxic effect on other cells (FIG. 1 & FIG. 2).

B. Mouse Spleen Cell Culture

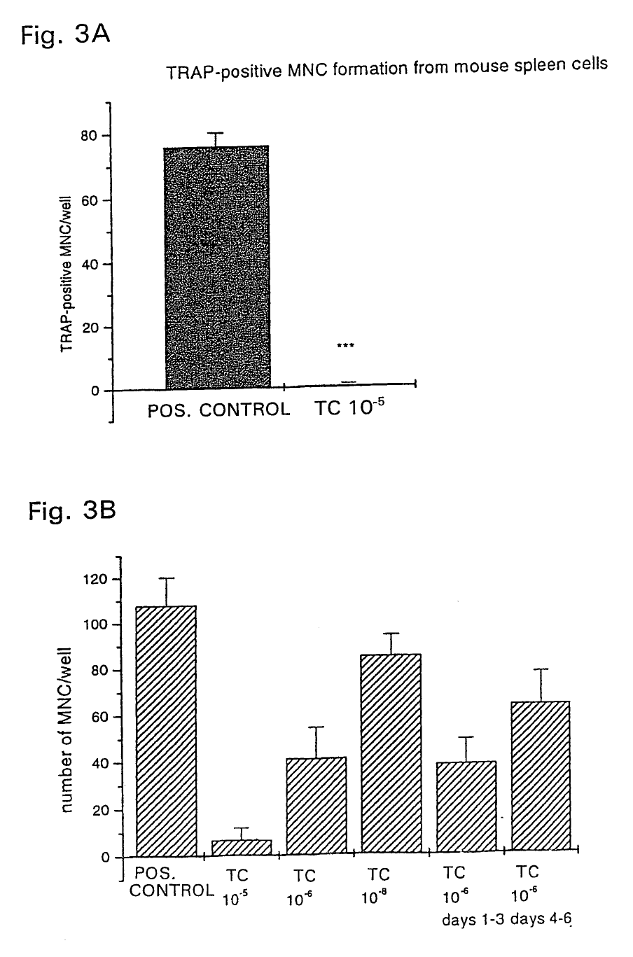

The test compound also dose dependently inhibited osteoclast formation in spleen cell culture. 60% inhibition was obtained at 10⁻⁶ M concentration as in the marrow culture system. At 10⁻⁵ M it completely blocked the TRAP-positive MNC formation (FIG. 3). When the compound was present only at days 1-3, the inhibition of osteoclast formation was more than 60%, whereas when it was present on days 4-6, the inhibition was approximately 40% (FIG. 3). This suggests that the said compound is affecting the early steps of osteoclast maturation. Because no osteoblastic or stromal cells were present in the culture, it appears that the compound affects directly osteoclast precursors. The mechanism of action, however, still remains to be solved. However, at 10⁻⁵ M it totally blocked osteoclast-like cell formation and no TRAP-positive MNC or mononuclear cells were present in the culture (data not shown). This suggests that the compound inhibited osteoclast precursor cell differentiation before it started to express TRAP.

C. Mouse Spleen Cell/Osteoblast Coculture

When these two cell types were cultured in the presence of 1,25(OH)₂D₃, several osteoclasts were formed. Spleen cells alone with 1,25(OH)₂D₃ were not able to differentiate into osteoclasts. Mouse osteoprogenitor cells, which were able to support osteoclast formation, were incubated first with 10⁻⁵ M of the said test compound for 24 h and then these cells were used in the co-culture with mouse spleen cells. It was found that so-treated preosteoblasts supported osteoclast formation in the similar manner as non-treated preosteoblasts (data not shown). This further supports the idea, that the test compound has direct inhibitory effect on osteoclast precursors.

D. Rat Osteoclast Culture

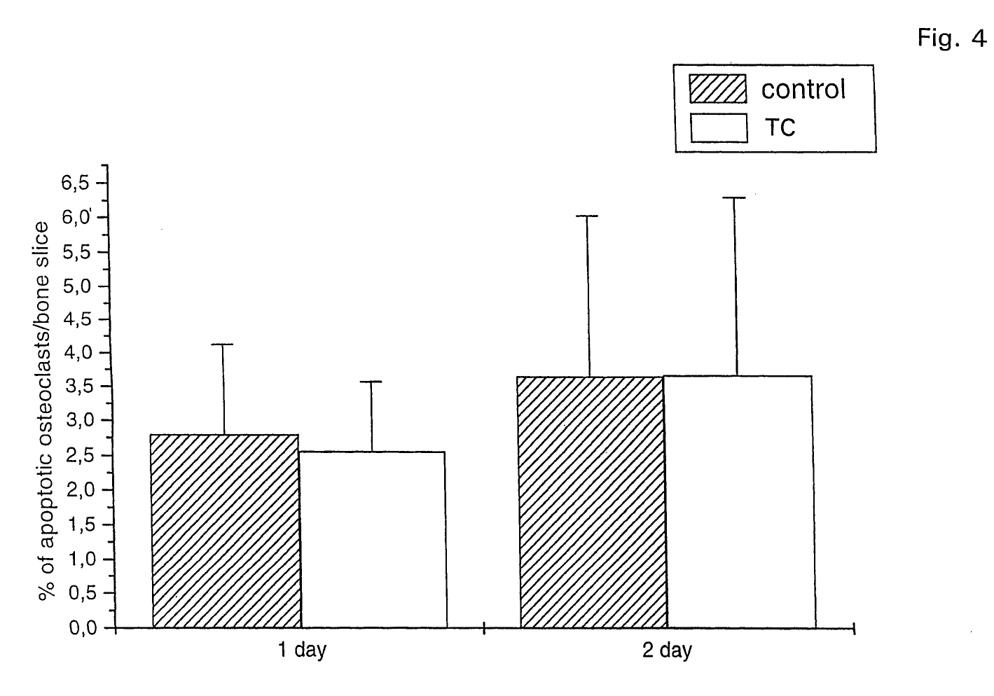

The compound did not have any effect on osteoclast-mediated bone resorption (data not shown). It did not have any effect on osteoclast apoptosis, when osteoclasts were cultured for 1-2 days on bovine bone slices (FIG. 4).

Tests on Ovariectomized Rats

The activity of [[(3-hydroxy-2-pyridyl)amino]methylidene]bisphosphonic acid tetraethylester (TC) has been tested in ovariectornized rats. In the said tests, the compound to be tested was administered to six-month old rats. The control rats were sham-operated or overiectomized and given vehicle. The compound was given to ovariectomized rats orally 7 days a week for 12 weeks beginning immediately after the surgery. The doses were 10, 50 and 100 mg/kg/day. At the end of the study, the mineral density and the geometry of the bone was measured with pQCT (peripheral quantitative computed tomography). The strength of the bone was measured with electromechanical materials testing machine. The sites of measurement were the proximal end of the tibia (not strength), the lumbar vertebra, the femoral neck and the tibial mid-diaphysis. The parameters to be measured were the bone density of the trabecular or cortical bone, the cortical area and strength of the bone.

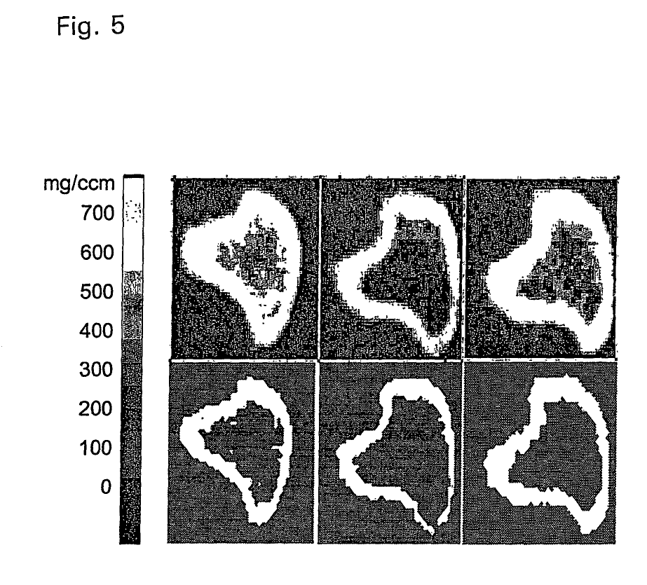

In the FIG. 5 the pQCT images are shown. In the upper row, from left to right, there is shown the bone mineral densities of a cross-section of the proximal tibia of a normal (sham-operated) rat, of an ovariectomized rat, and of an ovariectomized rat treated with the compound to be tested. From the results it is evident that the bone density in an ovariectomized rat is significantly reduced as compared to a normal rat, and also that this change is effectively inhibited by the compound tested. The lower row shows the corresponding measuring results for the cortical bone for a normal rat, an ovariectomized rat, and for an ovariectomized rat treated with the compound to be tested (counted from left to right). As can be seen, ovariectomy clearly reduces the thickness of the cortical bone as compared to the normal situation, which change can be inhibited with the compound tested.

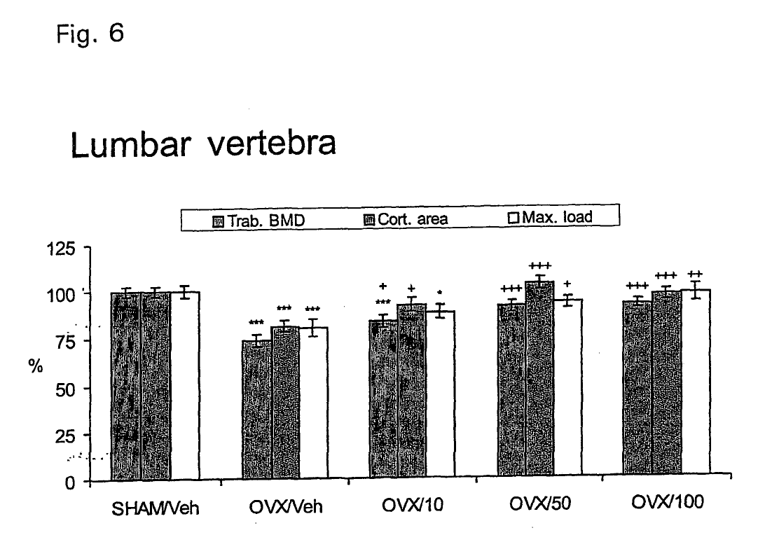

FIG. 6 shows that ovariectomy reduces the density of the trabecular bone and the area and strength of the cortical bone in the lumbar vertebra. The two higher doses of the tested compound, namely 50 and 100 mg/kg/day, inhibit these changes.

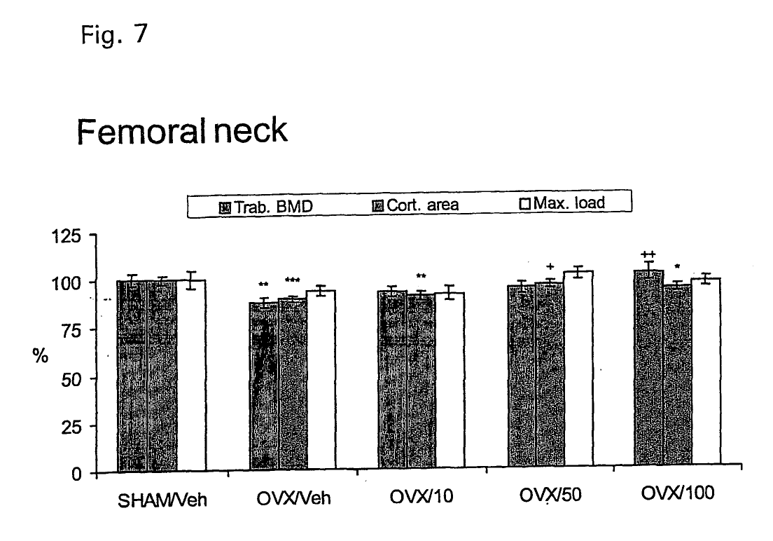

FIG. 7 in turn shows that 50 mg/kg/day of the compound tested inhibited the change in the cortical area in the femoral neck caused by ovariectomy; a dose of 100 mg/kg/day of the compound tested inhibited the change in the trabecular density.

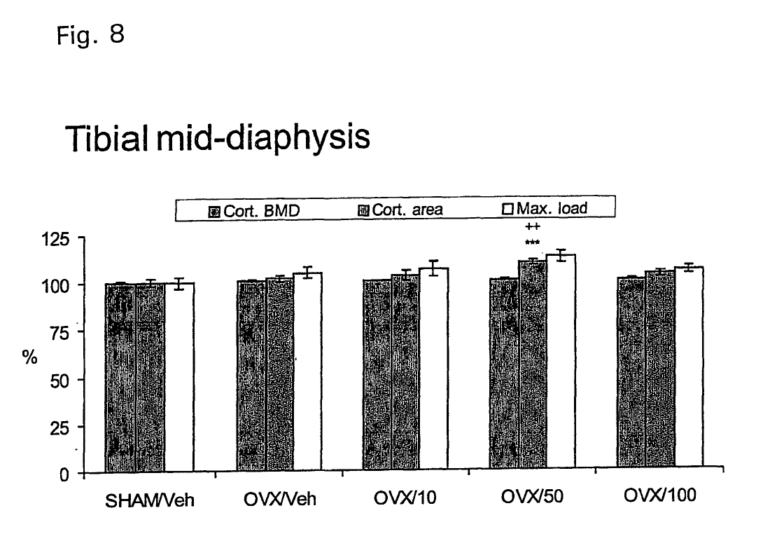

In the tibial mid-diaphysis, ovariectomy did not cause substantial changes, however, a dose of 50 mg/kg/day results in an increase of the cortical area as compared to a normal rat and an ovariectomized rat. Also the strength improved, although not to a significantly meaningful degree (FIG. 8).

The above results show that the beneficial effect on cortical bone is obtained in accordance with the invention, and that the compounds affect the osteoclast formation process rather than mature osteoclasts.

Tests in a Metastasis Model in Mouse

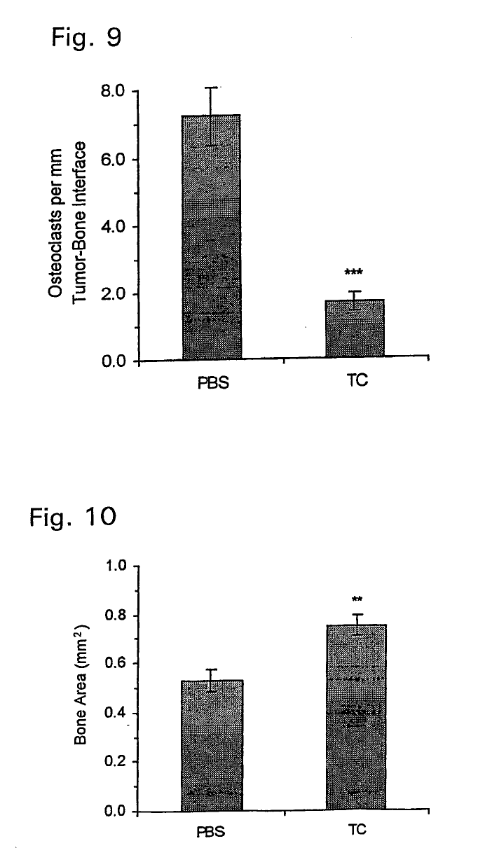

The effect of the test compound has been tested in a metastasis model in the mouse. 14 days after inoculation with human breast cancer cells, when the radiologically detected osteolytic bone lesions were found, the animals were treated either with PBS (control) or the test compound (50 mg/kg/day, s.c.) for 10 days. At the end of the study, osteoclast number and bone area were measured histomorphometrically. The test compound decreased significantly the number of osteoclasts at the tumor-bone interface as compared to the control (FIG. 9). Treatment with the test compound inhibited also tumor induced decrease in bone area at the site of metastases (FIG. 10).

### EXAMPLE 1

A pharmaceutical composition for oral use in the form of a tablet can be prepared using the following ingredients (per tablet)

| | |
|---|---|
| [[(3-hydroxy-2-pyridinyl)amino]methylene]bisphosphonic | 40.0 mg |
| acid tetraethyl ester | |
| Microcrystalline cellulose | 20.0 mg |
| Lactose | 67.0 mg |
| Starch | 10.0 mg |
| Talc | 4.0 mg |
| Magnesium stearate | 1.0 mg |

The ingredients are compounded into a tablet using conventional tablet making methods. If desired, the tablet may be coated with an enteric coating to control the point of release of the active agent.

### EXAMPLE 2

A pharmaceutical composition in the form of a capsule can be prepared using the following ingredients (per capsule)

| | |
|---|---|
| [[(3-hydroxy-2-pyridinyl)amino]methylene]bisphosphonic | 10.0 mg |
| acid tetraethyl ester | |
| Starch | 20.0 mg |
| Magnesium stearate | 1.0 mg |

The ingredients are combined in a known manner to form a mixture to be filled in a capsule of suitable size.

References

Arnett T R, Dempster D W (1987) A comparative study of disaggregated chick and rat osteoclasts in vitro: Effects of calcitonin and prostaglandins. Endocrinology 120: 602-608.

Boyde A, All N N, Jones S J (1984) Resorption of dentine by isolated osteoclasts in vitro. Br Dent J 156: 216-220.

Chambers T J, Revell P A, Fuller K, Athanasou N A (1984) Resorption of bone by isolated rabbit osteoclasts. J Cell Sci 66: 383-399.

Collins D A, Chambers T J (1992) Prostaglandin E2 promotes osteoclast formation in murine hematopoietic cultures through an action on hematopoietic cells. J Bone Miner Res 7: 555-561.

Hentunen T A, Reddy S V, Boyce B F, Devlin R, Park H-R, Chung H, Selander K S, Dallas M, Kurihara N, Galson D L, Goldring S R, Koop, B A Windle J J, Roodman G D (1998) Immortalization of osteoclast precursors by targeting bcl-XL and simian virus 40 large T antigen to the osteoclast lineage in transgenic mice. J Clin Invest 102: 88-97.

Jimi E, Nakamura I, Duong L T, Ikebe T, Takahashi N, Rodan G A, Suda T (1999) Interleukinl induces multinucleation and bone-resorting activity of osteoclasts in the absence of osteoblasts/stromal cells. Exp Cell Res 247: 84-93.

Lakkakorpi P, Tuukkanen J, Hentunen T, Järvelin K, V\ddot{aa}nänen H K (1989) Organization of osteoclast microfilaments during the attachment to bone surface in vitro. J Bone Miner Res 4: 817-825.

Owen M, Friendenstem A J (1988) Stromal stem cells: Marrow-derived osteogenic precursors. Ciba Found Symp 136:42-60.

Qu Q, Perälä-Heape M, Kapanen A, Dahllund J, Salo J, V\ddot{aa}nänen, H K, Härkönen, P (1988) Estrogen enhances differentiation of osteoblasts in mouse bone marrow culture. Bone 22:201-209.

Selander K, Lehenkari P, V\ddot{aa}nänen H K (1994) The effects of bisphosphonates on the resorption cycle of isolated osteoclasts. Calcif Tissue Int 55: 368-375.

Shinar D M, Sato M, Rodan G A (1990) The effect of hemopoietic growth factors on the generation of osteoclast-like cells in mouse bone marrow cultures. Endocrinology 126: 1728-1735.

Takahashi N, Yamana H, Yoshiki S, Roodman G D, Mundy G R, Jones S J, Boyde A, Suda T (1988a) Osteoclast-like cell formation and its regulation by osteotropic hormones in mouse bone marrow cultures. Endocrinology 122:1373-1382.

Takahashi N, Akatsu T, Sasaki T, Nicholson G C, Moseley J M, Martin T J, Suda T (1988b) Induction of calcitonin receptors by 1α, 25-dihydroxyvitamin D3 in osteoclast-like multinucleated cells formed from mouse bone marrow cells. Endocrinology 123: 1504-1510.

Udagawa N, Takahashi N, Akatsu T, Sasaki T, Yamaguchi A, Kodama H, Martin T J, Suda T (1989) The bone marrow-derived stromal cell lines MC3T3-GA/PA6 and ST2 support osteoclast-like cell differentiation in cocultures with mouse spleen cells. Endocrinology 125: 1805-1813.

1. Use of a compound of the formula 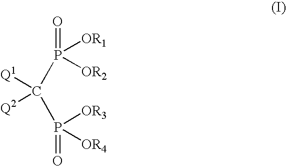
in which formula
Q¹ is hydrogen, hydroxyl, halogen, amino —NH₂, or —OR′, wherein R′ is C₁-C₄-alkyl or acyl,
Q² is
a) straight or branched C₁-C₁₀-alkyl, -hydroxyalkyl or -aminoalkyl, or C₂-C₁₀-alkenyl, -hydroxyalkenyl or -aminoalkenyl, or -alkynyl, -hydroxyalkynyl or -aminoalkynyl, whereby the oxygen may, as a substituent, contain one protecting group, and the nitrogen, as a substituent, may contain one or two protecting groups, which are especially C₁-C₆-alkyl, acyl, aryl or arylalkyl, or Q² is unsubstituted or substituted, saturated or unsaturated C₃-C₁₀-cycloalkyl, which can be bound to the methylene carbon over a straight or branched C₁-C₄-alkylene group, or
b) the group 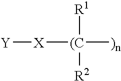
wherein Y is a saturated, partly saturated or an aromatic heterocyclic group, or a carbocyclic aromatic group, whereby the heterocyclic group can contain 1 to 3 hetero atoms from the group N, O and S, X is a bond, O, S or NR³, wherein R³ is hydrogen or a protecting group, especially C₁-C₆-alkyl, acyl, aryl or arylalkyl, and n is an integer 0 to 6, R₁ and R₂ are independently hydrogen or C₁-C₄-alkyl, or
c) halogen,
and the groups R₁ to R₄ are the same or different, and mean an alkyl group with 1-5 carbon atoms, whereby one or two of the groups R₁ to R₄ can also mean hydrogen, or a pharmacologically acceptable salt thereof, for the preparation of a pharmaceutical composition having osteoclast formation inhibiting or suppressing activity.

2. The use according to claim 1, wherein in the compound of the formula I, Q¹ is hydrogen or hydroxyl.

3. The use according to claim 1 or 2, wherein the compound of the formula I is a tri- or tetraester, preferably a tetraester.

4. The use according to any preceding claim, wherein in the compound according to the formula I Q² is C₁-C₆-alkyl or hydroxy- or amino-substituted C₁-C₆-alkyl, or C₁-C₆-alkyl-mono- or -di-substituted amino-C₁-C₆-alkyl, especially -C₁-C₄-alkyl.

5. The use according to claim 4, wherein Q² is methyl, ethyl, propyl, isopropyl, straight or branched butyl or pentyl, or the corresponding hydroxy- or amino-substituted group, such as 2-hydroxy- or 2-amino-ethyl, 3-hydroxy- or 3-amino-propyl, 4-hydroxy- or 4-amino-butyl, 5-hydroxy- or 5-amino-pentyl.

6. The use according to claim 1, wherein in the compound of the formula I there is present at least one heteroatom selected from the group of O, N and S, either in the ring Y and/or in the bridging group X.

7. The use according to claim 6, wherein Y is a nitrogen containing heterocyclic group, such as pyridinyl, pyrimidinyl or piperidinyl, which can be substituted with 1 to 3 substituents selected from hydroxy, protected hydroxy, C₁-C₄-alkyl, C₁-C₄-aloxy, halogen, amino, protected amino and/or nitro.

8. The use according to claim 1, wherein a compound having the formula I′ 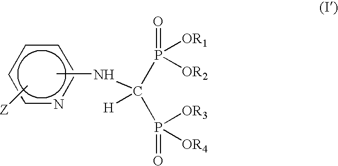
is used, in which formula Z has the meaning of hydroxy, or protected hydroxy, and the groups R₁ to R₄ are the same or different, and mean an alkyl group with 1-5 carbon atoms, whereby one of the groups R₁ to R₄ can also mean hydrogen.

9. The use according to claim 8 wherein Z is hydroxy.

10. The use according to claim 8, characterized in that the active agent is [[(3-hydroxy-2-pyridinyl)amino]methylidene]bisphosphonic acid tetraethylester.

11. The use according to any of the preceding claims for the preparation of a pharmaceutical composition with an effect strengthening bone or combating weakening of bone.

12. The use according to claim 11 for the preparation of a pharmaceutical composition with an effect strengthening cortical bone or combating weakening of cortical bone.

13. Method for treating an individual in need of a treatment for strengthening bone or for combating weakening of bone, comprising administering to such an individual an effective amount of a compound of formula I as defined in any of the preceding claims 1 to 10.

14. The method according to claim 13, comprising administering [[(3-hydroxy-2-pyridinyl)amino]methylidene]bisphosphonic acid tetraethylester.

15. The method according to claim 13 or 14 comprising administering the compound of formula I as defined in any of the preceding claim 1 to 10 in combination with other bone active treatments including, but not limited to, bisphosphonates and/or other specific estrogen receptor modifying agents.

16. The method according to any one of claims 13, 14 or 15, wherein the individual in need of said treatment is a postmenopausal female, a child, an elderly male, a patient treated with corticosteroids, cancer chemotherapies or other bone strength weakening therapies, a patient with cancer metastases in the bone, or a cancer patient whose bone strength is weakened by local or systemic factors secreted by the cancer cells.

17. The method according to claim 16 wherein the individual in need of said treatment is a postmenopausal female or a child.
